(19) United States
(12) Patent Application Publication
(10) Pub. No.: US 2011/0098374 A1
(43) Pub. Date: Apr. 28, 2011

(54) BONE CEMENT MIXTURE FOR PRODUCING AN MRT-SIGNALING BONE CEMENT

(76) Inventors: Florian Wichlas, Berlin (DE); Hermann Josef Bail, Wendelstein (DE); Ulf Teichgräber, Berlin (DE); Jens Pinkernelle, Berlin (DE); René Schilling, Brandenburg (DE)

(21) Appl. No.: 12/867,210

(22) PCT Filed: Feb. 12, 2009
(86) PCT No.: PCT/EP09/01201

§ 371 Date: Dec. 17, 2010

(30) Foreign Application Priority Data
Feb. 12, 2008 (DE) 10 2008 009 218.5
Mar. 10, 2008 (DE) 10 2008 013 620.4

(51) Int. Cl. A61F 2/28

(52) U.S. Cl. 523/116

(57) ABSTRACT

The invention relates to a bone cement mixture for producing an MRT-signaling bone cement and to an MRT-signaling bone cement produced from said mixture. The bone cement mixture of the invention includes:
(a) at least one type of polymerizable organic monomers and optionally at least one already polymerized polymer component;
(b) at least one MRT-signaling component with a magnetic susceptibility and a concentration in the cement mixture sufficient to generate a visible signal in at least one selected MRT sequence; and
(c) at least 1 wt. % water, based on the total mass of the bone cement mixture.

The bone cement mixture according to the invention is remarkable for its good signals in MRT in at least one measuring sequence, especially in the T1 sequence.

The invention relates to a bone cement mixture for producing an MRT-signaling bone cement and to a polymer-based bone cement produced from said bone cement mixture.

Bone cements for filling bone fractures or as bone replacement material are well-known, e.g. following surgical intervention in cases of bone cancer; they are incorporated in a defect site having formed to harden therein and replace the natural bone material. Other fields of application of bone cements are anchoring of endoprostheses in major joints, support of implants (plates, nails, etc.) in osteoporotic fractures as composite osteosynthesis, or the use as placeholder in infected joints. A relatively new surgical technique which uses bone cement is, for example, vertebroplasty, or kyphoplasty, wherein a vertebral body broken e.g. as a result of osteoporosis is stabilized by injecting bone cement.

Conventional bone cements are based on inorganic materials, such as calcium phosphate, which harden after mixing with water as a result of setting. More recent bone cements are based on organic polymers, in which event the bone cement mix includes polymerizable monomers and an initiator and/or activator to initiate polymerization, so that solidification takes place by way of cold polymerization. For example, a common organic bone cement is based on polymethyl methacrylate (PMMA) which is obtained by polymerizing the methyl methacrylate (MMA) monomer. Commercial PMMA bone cements are marketed in the form of two-component systems which have to be mixed. The liquid component contains MMA as major component and usually an activator (e.g. N,N-dimethyl-p-toluidine) and/or a stabilizer/inhibitor (hydroquinone) to prevent polymerization during storage. The powder component consists mainly of particulate PMMA polymers, frequently already added with an initiator (e.g. benzoyl peroxide) to initiate free-radical polymerization after mixing the two components. In addition, the powder component may include an X-ray contrast agent (e.g. zirconium dioxide, barium sulfate) and/or an antibiotic agent and/or a dye. Instead of the pure polymers of MMA, it is also well-known to use copolymers with MMA and a comonomer or mixtures of PMMA and MMA copolymers. The powdered polymer component is primarily used to achieve a sufficiently high viscosity of the mixture for processing. A review on bone cements based on PMMA can be found in Breusch & Kühn, Orthopädie 32 (2003), 41-50.

With magnetic resonance tomography (MRT), a diagnostic imaging method has been established in medicine in recent decades, by means of which sectional images of tissue structures can be generated at a defined tissue depth in the human body. Unlike in computed tomography (CT), wherein mainly solid, X-ray-impermeable structures such as bones are visualized, the potential of MRT is essentially based on visualizing water-containing tissue structures, so that MRT is particularly suitable for assessing organs. As MRT utilizes magnetic fields and electromagnetic waves, one advantage can be seen in the absence of radiation exposure of patients and medical staff. More recent developments are concerned with the above-mentioned imaging in a so-called “open MRT” wherein surgery is performed together with MRT monitoring.

The MRT measuring method is based on the alignment of atomic nuclei of hydrogen in a strong static electromagnetic field, which have a magnetic moment as a result of their intrinsic angular momentum (spin). When applying a second, high-frequency alternating field (transverse field) at a right angle to the first field, the nuclei will be disturbed in their original position and begin a precession movement, during which—in a simplified representation—their axes of nuclear rotation are in a tilted orientation compared to that in a static field. Which nuclei will undergo resonance is determined by selecting the strength of the static field and the frequency of the transverse field. In MRT, these are basically the hydrogen nuclei of water. After switching off the transverse alternating field, the nucleus will further precess for some relaxation time in the original plane defined by the alternating field until it falls back to its thermal equilibrium. As a result, a tissue type-dependent transverse magnetization is generated to induce a current flow in a coil of the tomograph, which represents the actual measured quantity.

MRT uses different measurement sequences which differ in the frequency of the transverse alternating field and/or the strength of the static magnetic field. T1-based measuring frequencies are used to measure the spin-lattice relaxation (longitudinal relaxation) and emphasize the visualization of solids. In contrast, T2-based measuring frequencies are used to measure the spin-spin relaxation (transverse relaxation) and provide particularly good visualization of soft tissues. To facilitate the overall assessment of all structures existing in the body, the visualization of an organ therefore generally proceeds using both T1 and T2 sequences.

Conventional bone cements involve the drawback of being invisible in magnetic resonance imaging (MRI) and can only be identified through the absence of signals. For this reason, differentiation from other non-signaling structures is practically impossible because the absence of a signal does not allow reliable conclusions as to the presence of the cement. An MRT-signaling bone cement would be desirable for subsequent control of surgically incorporated patches of bone cement and assessment of surrounding structures in an MRT. An MRT-visible bone cement is indispensable especially in modern surgical techniques performed under MRT monitoring (“open MRT”), for instance in the above-mentioned vertebroplasty, or when filling surgically cleared tumor cavities with bone cement under MRT monitoring.

As mentioned above, it is well-known to add an X-ray contrast agent to the PMMA cement, in particular the PMMA-containing powder component, so as to allow visualization of the bone cement in X-ray diagnostics or computerized tomography. An inorganic, calcium phosphate-based bone cement is known from US 2005/0287071 A1, which is visible in X-ray images as a result of adding an X-ray opaque material.

U.S. Pat. No. 6,585,755 B2 describes an endovascularly implantable object, in particular a stent, made of an organic polymer material added with an MRT additive so as to make the stent, which permanently remains in the body, “MRT-compatible”, that is, prevent or compensate interactions with the MRT magnetic field that are caused by the stent. However, direct MRT visibility of the stent in terms of active signaling cannot be assumed.

An MRT-visible bone cement is not known as yet.

The invention is therefore based on the object of providing a bone cement mixture for producing a bone cement based on organic polymers, which produces a sufficient signal in MRT to be visible therein.

Said object is accomplished by means of a bone cement mixture and a bone cement which is produced from said mixture and has the features according to the independent claims. The inventive bone cement mixture used to produce an MRT-signaling bone cement includes
(a) at least one type of polymerizable organic monomers and optionally at least one already polymerized polymer component;
(b) at least one MRT-signaling component with a magnetic susceptibility and a concentration in the cement mixture sufficient to generate a visible signal in at least one selected MRT sequence; and
(c) at least 1 wt. % water, based on the total mass of the bone cement mixture.

It was found that the bone cement according to the invention is well visualizable in MRT by generating a well-differentiable signal in said at least one MRT sequence. This result was surprising in so far as the hardened cement is a solid per se invisible in MRT which basically visualizes protons from water. While conventional bone cements will also become saturated with water inside the body to a certain level, this level is not sufficient to generate a signal. Most surprisingly, the combination of water and MRT-signaling component (hereinafter also referred to as MRT contrast agent) is indispensable for generating a signal in the cement in an MRT in a synergistic manner. By suitably selecting the type and concentration of the signaling component and the proportion of water, it is also possible to successfully generate differentiable signals of the cement in different measuring sequences so as to allow an overall assessment of the organ under investigation.

More specifically, the three components (a), (b) and (c) specified above constitute at least 80 wt. %, preferably at least 90 wt. %, and in general even at least 95 wt. % of the total mixture. Preferably, the cement mixture according to the invention consists essentially of these components. The remaining weight percentage may optionally comprise auxiliary materials such as X-ray contrast agents, polymerization initiators, organic solvents for the monomers, etc.

In a preferred embodiment the water content is selected such that the cement will be visible in at least one sequence. Care should be taken that, on the one hand, sufficient water saturation in the cement is obtained, so that the signal of the MRT contrast agent is sufficient, and, on the other hand, the stability and workability of the cement is not impaired as a result of an excessively high proportion of water. In a preferred fashion the maximum possible water content with respect to cement stability is selected. Mass percentages of water of from 10 to 60%, in particular of from 5 to 45%, preferably from 14 to 23%, based on the total mass of the mixture, have proven beneficial.

Furthermore, it is preferably envisaged to select the magnetic susceptibility and the concentration of said at least one MRT-signaling component in the cement mixture in such a way that the cement generates a signal in said at least one sequence, particularly in the T1 sequence or in a T1-based sequence. There is an interaction between susceptibility and concentration such that with increasing susceptibility the required concentration of MRT contrast agent is lower and vice versa. The lower concentration limit is selected in such a way that sufficient signal is obtained, whereas the upper concentration limit is dimensioned such that no signal quenching occurs. In this context, a person skilled in the art is familiar with the practice of deriving materials of suitable magnetic susceptibility from specialist tables and, if necessary, experimentally determining the suitable concentration using simple series of mixtures. It will be appreciated that the selection of the MRT-signaling component and the concentration thereof in the cement mixture must be adapted to the magnetic field strength of the MRT instrument to be used, and the lower the strength of the instrument, the higher the required susceptibility and/or concentration. In general, current MRT instruments have magnetic field strengths of 1 Tesla or more.

In particular, paramagnetic or ferromagnetic metals are possible as MRT-signaling component and can be in metallic form, in the form of a compound, salt and/or complex. Said component is preferably selected from transition metals (in particular the fourth period from scandium to zinc), lanthanides and alkaline earth metals (particularly magnesium and calcium), as well as compounds, salts and complexes thereof. In selecting the contrast agent, care should be taken that it has lowest possible toxicity, low migration tendency and—in the event of a compound or complex—a safe metabolic pathway. However, owing to the extremely low concentration of contrast agent required, the aspect of toxicity is of minor importance. Particularly preferred MRT-signaling components comprise manganese, iron, cobalt, nickel, copper, chromium, titanium, vanadium, scandium, zinc, gadolinium, dysprosium, as well as calcium and magnesium, and these elements can be used in the form of metals or alloys, compounds, complexes or salts. In principle, all transition metals or materials with ferromagnetic or paramagnetic properties can be used.

According to another advantageous embodiment of the invention, a mixture of two or more MRT-signaling components is used, which components generate signals in different measuring sequences.

In a preferred fashion the metal of the at least one MRT-signaling component is present only in traces in the total mixture. Its concentration, based on the total mass of the cement mixture, is typically from 0.05 to 5 μmol/g, particularly from 0.1 to 3 μmol/g. The lower concentration limit is selected in such a way that sufficient signal is obtained in MRT, whereas the upper concentration limit is dimensioned such that no signal quenching occurs. Typical mass percentages range from a few 10⁻³ wt. %, for instance from 0.1·10⁻³ to 40·10⁻³ wt. %. All the above figures refer to the pure metal, also including its use in the form of a compound or complex.

It is preferably envisaged that the MRT contrast agent is distributed in the cement mixture as homogeneously as possible. Especially in the event of metals or alloys, it can be distributed homogeneously in the form of, for example, nano- or microparticles in the cement. In the event of insoluble compounds or complexes, a homogeneous dispersion is the option of choice, and with soluble compounds or complexes, a homogeneous solution in the cement mixture is possible.

An acrylate-based polymer is preferred as cement base material, although the invention is not limited thereto. Accordingly, said at least one type of polymerizable organic monomers is selected from the group of acrylates, in particular comprising methacrylate, methyl methacrylate, ethyl acrylate, ethyl methacrylate, propyl acrylate, propyl methacrylate, butyl acrylate and butyl methacrylate, preferably methyl methacrylate (MMA), the polymerization of which results in polymethyl methacrylate (PMMA) or an MMA-containing copolymer. Similarly, an acrylate-based polymer or copolymer is preferred as said already polymerized polymer component, in particular based on acrylates comprising methacrylate, methyl methacrylate, ethyl acrylate, ethyl methacrylate, propyl acrylate, propyl methacrylate, butyl acrylate and butyl methacrylate. In a particularly preferred fashion the polymer component is PMMA or a copolymer of MMA and butyl methacrylate. Apart from organic polymer cements, the principle according to the invention can basically be applied to any curable or settable material offering the option of including an MRT contrast agent and water. Thus, any conventional inorganic cement, especially calcium-based cements or hydroxylapatite, can be used instead of said organic polymer.

While polymerization can be initiated by heat or radiation sources, it is preferred for physiological and practical reasons to use a (cold) polymerization that is triggered by a free-radical initiator which is included in the cement mixture and, after combining the individual components, initiates polymerization in particular via formation of free radicals. Accordingly, the cement mixture includes at least one initiator and/or activator to induce polymerization of the monomers.

Further, quantitatively minor components may of course be present in the cement mixture, especially stabilizer(s) to prevent polymerization during storage of the monomers, dye(s) for staining the hardened cement, antibiotic agents for release from the cement and/or X-ray contrast agents for visualization of the cement in an X-ray instrument or in CT.

A cement mixture in the meaning of the present invention is not only understood to be a composition of the previously mixed components. The cement mixture is preferably in the form of a kit in separate components to be mixed by the user immediately prior to use so as to initiate polymerization. More specifically, the kit comprises at least one liquid phase, which contains at least the monomers, and a solid phase which contains the, in particular powdered, polymer component. According to a specific embodiment, the kit comprises two liquid phases, with a first liquid phase (non-aqueous, hydrophilic phase) containing the monomers and a second liquid phase (aqueous phase) containing said at least one MRT-signaling component and the water. In a preferred fashion these phases are initially emulsified with each other prior to mixing the emulsified mixture with the solid phase.

The MRT-signaling bone cement prepared from the bone cement mixture according to the invention is obtained by polymerizing the at least one type of polymerizable organic monomers, so that the product contains no or only minor amounts of unreacted monomers but instead the polymer product thereof.

Accordingly, a method for repairing bone defects comprises the steps of producing the bone cement mixture according to the invention by intense mixing of the individual components with each other, incorporating the bone cement mixture in the site of the defect and curing the mixture by polymerizing the monomers. In this context, a bone defect is understood to be any bone abnormality of a bone, especially fractures and abnormal positions, e.g. as a result of surgical intervention. Incorporating the bone cement mixture in the site of the defect is preferably performed in a so-called open MRT, i.e. under direct MRT monitoring.

Other preferred embodiments of the invention can be inferred from other features specified in the subclaims.

The invention will be explained below in examples with reference to the accompanying drawings wherein

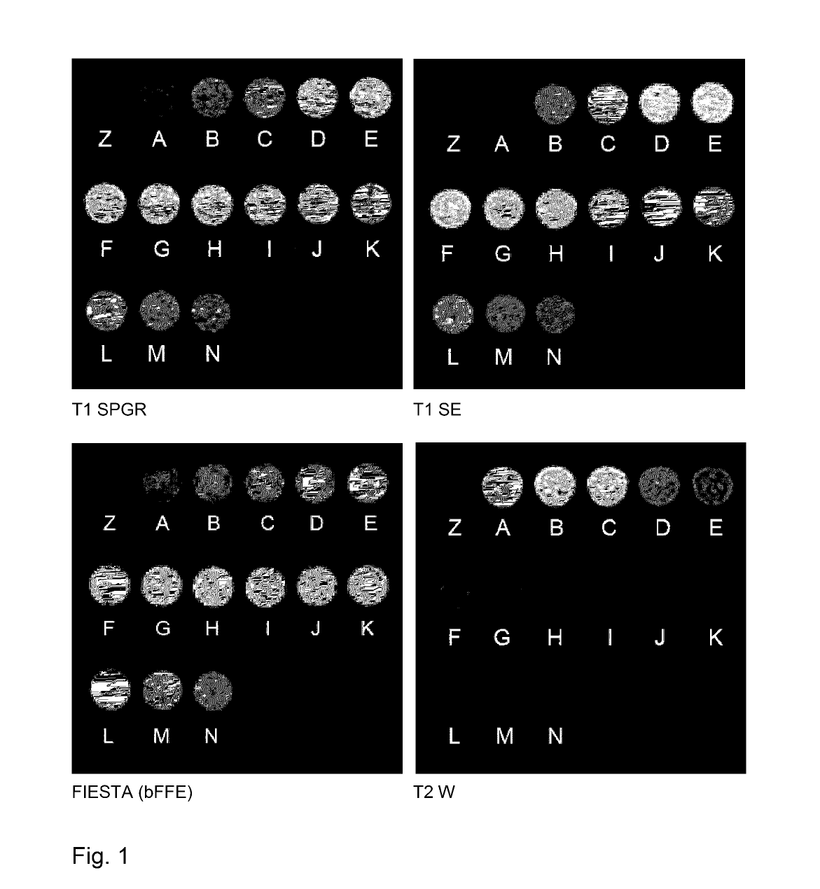

FIG. 1 shows PMMA bone cement samples with varying concentrations of a Gd complex (gadobenic acid meglumine), visualized with different MRT sequences;

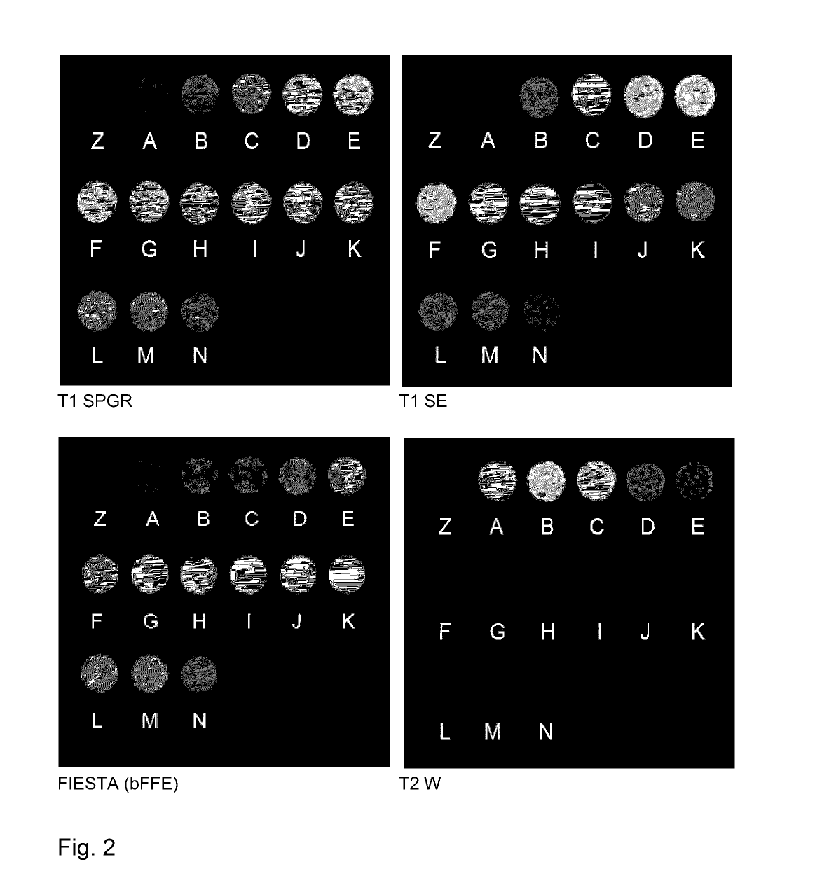

FIG. 2 shows PMMA bone cement samples with varying concentrations of a Gd complex (gadobenic acid dimeglumine salt), visualized with different MRT sequences;

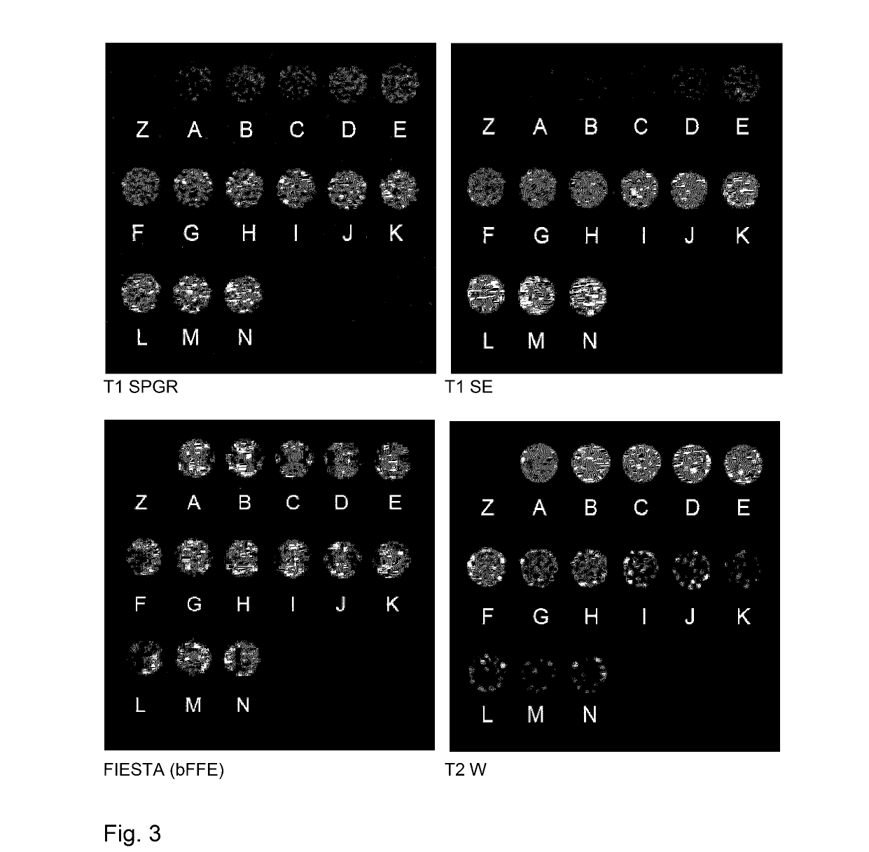

FIG. 3 shows PMMA bone cement samples with varying concentrations of an Mn complex (mangafodipir trisodium), visualized with different MRT sequences;

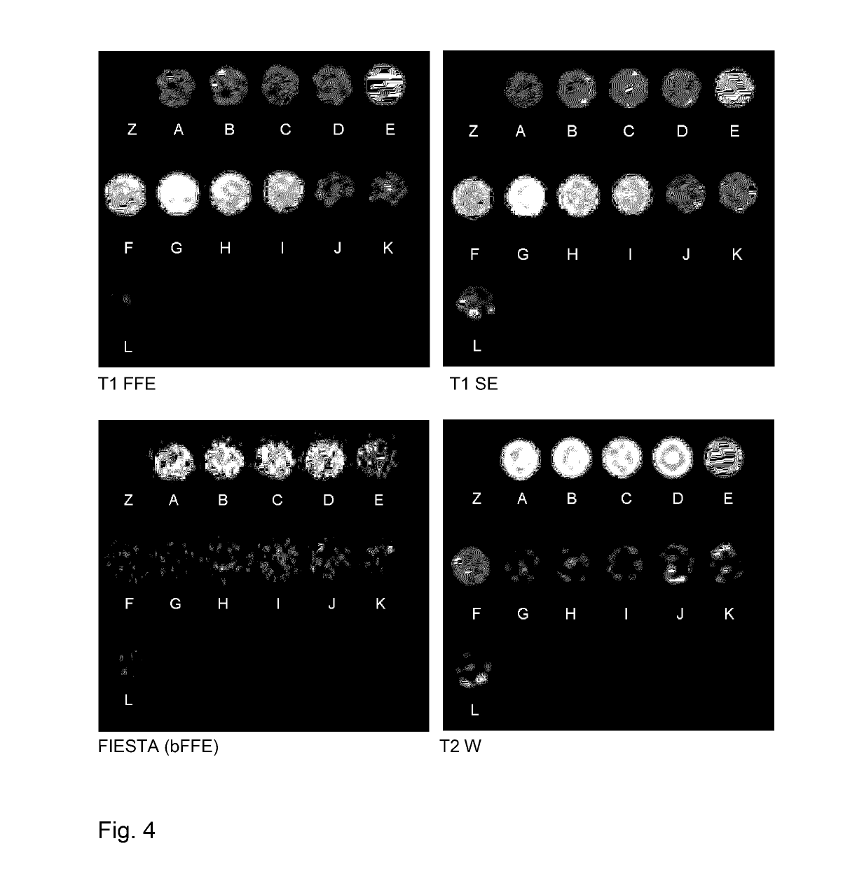

FIG. 4 shows PMMA bone cement samples with varying concentrations of iron oxide nanoparticles, visualized with different MRT sequences;

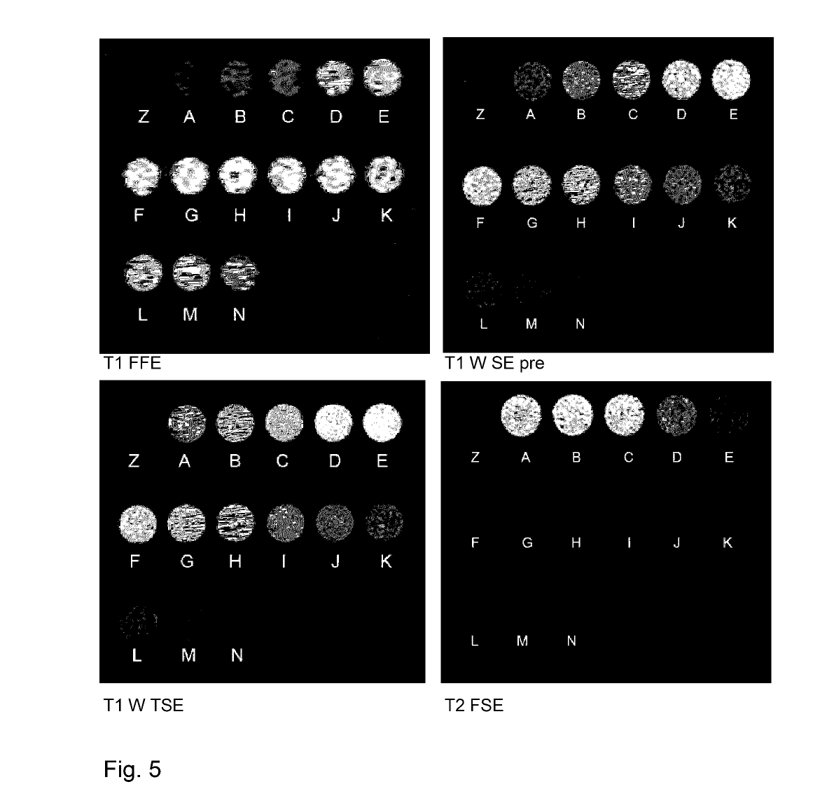

FIG. 5 shows PMMA bone cement samples with varying concentrations of manganese(II) chloride tetrahydrate, visualized with different MRT sequences;

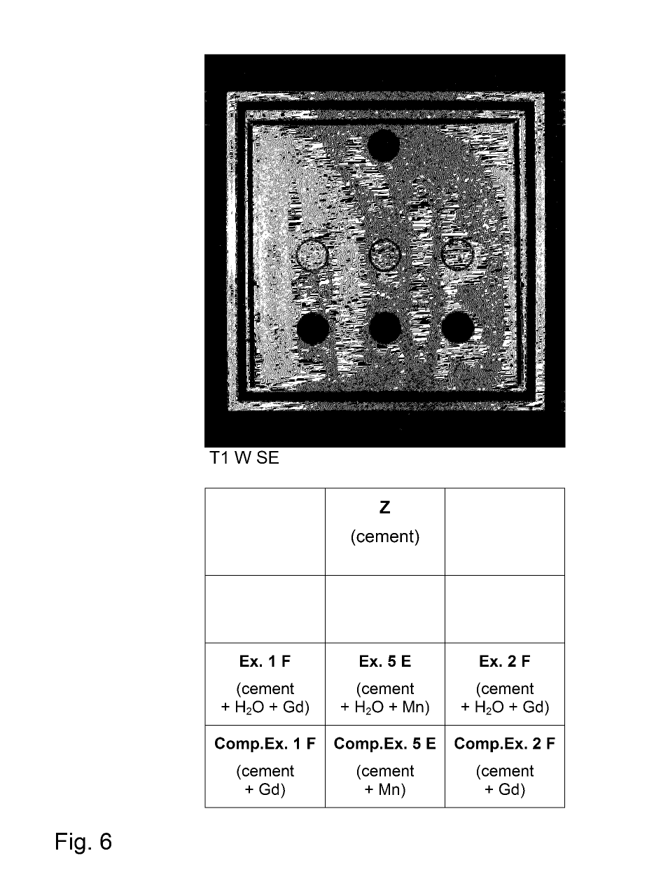

FIG. 6 shows PMMA bone cement samples with different MRT contrast agents with and without addition of water;

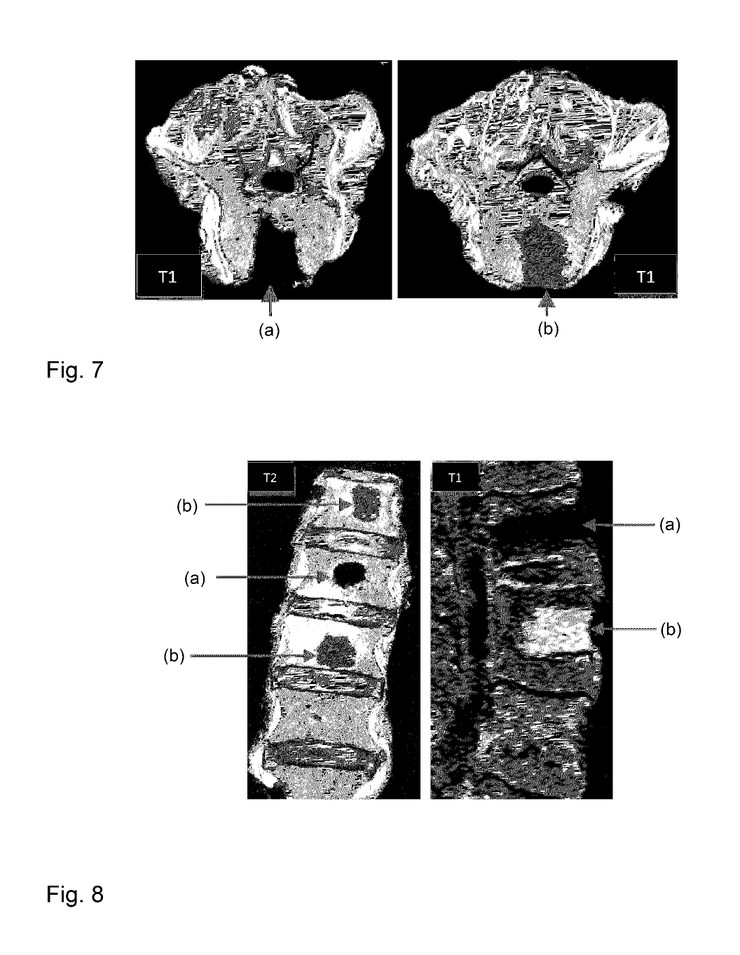

FIG. 7 shows a transverse MRT representation of a vertebral body with a fracture filled with a conventional PMMA bone cement (left) and an inventive PMMA bone cement including gadobenic acid meglumine as contrast agent (right); and

FIG. 8 shows a sagittal MRT representation of a spine in which vertebral bodies have been filled with a conventional PMMA bone cement (arrows (a)) and with an inventive PMMA bone cement including gadobenic acid meglumine as contrast agent (arrows (b)).

The preparation of the cement samples was basically performed by preparing the aqueous phase containing the MRT contrast agent and subsequently mixing the aqueous phase with the liquid cement component containing the monomer (specifically MMA). This liquid mixture was mixed with the powdered cement component containing the already polymerized material (specifically PMMA).

The cured cement samples were measured with the following MRT sequences and evaluated. It should be noted that the designations of the sequences involve information supplied by the manufacturer so that the designations on the instruments used (Philips Gyroscan, Philips Panorama, GE-MRT) may vary:
T1 (=T1 SE): spin echo in T1 with good resolution—assessment of solids (cement) and signal intensity
T1 FFE (T1=FSPGR): fast sequence in T1 weighting
T2 (=T2W): T2 weighting, fluids (water)
FIESTA (=bFTE, balanced FFE): fast gradient echo sequence.

### EXAMPLE 1

### PMMA-Cements Including Varying Concentrations of Gd

### Gadobenic Acid Meglumine

Chemicals:

| | |
|---|---|
| Dotarem ® (Guerbet GmbH) | 1 ml contains: |
| | 279.32 mg of gadobenic acid |
| | meglumine |
| | water for injection |
| 0.9 wt. % NaCl in H₂O | |
| MMA BonOs ® (AAP | 10 ml contain: |
| Implantate AG) | 9.93 ml of methyl methacrylate |
| | 0.07 ml of N,N-dimethyl-p-toluidine |
| | 60 ppm of hydroquinone |
| PMMA BonOs ® (AAP | 24 g of cement powder contain: |
| Implantate AG) | 10.95 g of polymethyl methacrylate |
| | 10.80 g of zirconium dioxide |
| | 0.50 g of benzoyl peroxide |

Varying amounts of a 0.5 molar aqueous solution of gadobenic acid meglumine (Dotarem®, Guerbet GmbH) were initially pipetted into 10 ml of a 0.9% aqueous NaCl solution each time (mixture I). 5 ml of this mixture I was mixed with 5 ml of liquid methyl methacrylate (MMA BonOs®, AAP Implantate AG) each time, which already included 7 vol. % N,N-dimethyl-p-toluidine as activator and 60 ppm hydroquinone as initiator (mixture II). The total amount (10 ml) of this mixture II was mixed with 12 g of solid cement component (PMMA BonOs®, AAP Implantate AG), i.e. 5.475 g of PMMA, 5.40 g of ZrO₂ as X-ray contrast agent, and 0.25 g of benzoyl peroxide. In view of the poor miscibility of the hydrophobic polymer component with the aqueous phase, the suspensions were prepared with constant mixing and processed immediately. The components of the mixture are summarized in Table 1. The final samples contained between 0 wt. % or 0 μmol/g (sample A) and 0.12 wt. % or 7.4 μmol/g (sample N) gadolinium (Gd) and about 22.7 wt. % H₂O each time.

TABLE 1

| | Mixture I | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Dotarem ® | 0 | 5 | 10 | 30 | 50 | 100 | 150 | 200 | 250 | 300 | 400 | 500 | 600 | 700 |
| [μl] | | | | | | | | | | | | | | |
| NaCl 0.9% | 10 | 10 | 10 | 10 | 10 | 10 | 10 | 10 | 10 | 10 | 10 | 10 | 10 | 10 |
| [ml] | | | | | | | | | | | | | | |
| | Mixture II | | | | | | | | | | | | | |
| | 1a | 2b | 3c | 4d | 5e | 6f | 7g | 8h | 9i | 10j | 11k | 12l | 13m | 14n |
| Mixture I | 1 | 2 | 3 | 4 | 5 | 6 | 7 | 8 | 9 | 10 | 11 | 12 | 13 | 14 |
| 5 ml | | | | | | | | | | | | | | |
| BonOs ® | 5 | 5 | 5 | 5 | 5 | 5 | 5 | 5 | 5 | 5 | 5 | 5 | 5 | 5 |
| MMA [ml] | | | | | | | | | | | | | | |
| | Sample | | | | | | | | | | | | | |
| | A | B | C | D | E | F | G | H | I | J | K | L | M | N |
| Mixture II | 1a | 2b | 3c | 4d | 5e | 6f | 7g | 8h | 9i | 10j | 11k | 12l | 13m | 14n |
| 10 ml | | | | | | | | | | | | | | |
| BonOs ® | 12 | 12 | 12 | 12 | 12 | 12 | 12 | 12 | 12 | 12 | 12 | 12 | 12 | 12 |
| PMMA [g] | | | | | | | | | | | | | | |
| w(Gd) | | | | | | | | | | | | | | |
| [10⁻³%] | 0.00 | 0.89 | 1.79 | 5.34 | 8.89 | 17.7 | 26.4 | 35.0 | 43.6 | 52.1 | 68.7 | 85.1 | 101 | 117 |
| c(Gd) | | | | | | | | | | | | | | |
| [μmol/g] | 0.00 | 0.06 | 0.11 | 0.34 | 0.57 | 1.13 | 1.68 | 2.23 | 2.77 | 3.31 | 4.37 | 5.41 | 6.43 | 7.43 |

Cylindrical test specimens of cement samples were prepared by filling into disposable syringes and allowed to cure. The test specimens of samples A through N, together with a reference sample free of contrast agent and water (sample Z), which was prepared according to the manufacturer's instructions and carried along, were arranged on a screen and measured in MRT (Philips 1.5 Tesla MRT (CVK), open MRT Panorama 1.0 Tesla (CCM) or GE 3 Tesla Signa (CVK)), using a head coil and applying the MRT sequences specified above. The results are shown in FIG. 1. As can be seen, the reference sample Z gives no signal in any of the sequences, i.e. is invisible in MRT. Similarly, sample A containing water but no Gd shows only a very weak signal in the T1 sequences and a weak signal in the T2 W fluid-sensitive sequence and in the bFFE mixed sequence. In T1 FSPGR and T1 SE the samples D through H (0.34 to 2.23 μmol/g Gd) gave the strongest signals. In bFFE the strongest signals were determined in the samples E through K (0.57 to 4.37 μmol/g Gd). In T2 W the samples B and C (0.06 to 0.11 μmol/g Gd) gave the strongest signals. At even higher concentrations of Gd the signal decreases as a result of quenching.

### EXAMPLE 2

### PMMA Cements Including Varying Concentrations of Gd

### Gadobenic Acid Dimeglumine

Chemicals:

| | |
|---|---|
| Multihance ® | 1 ml contains: |
| (Bracco Altana Pharma GmbH) | 529 mg of gadobenic acid dimeglu- |
| | mine salt (334 mg of gadobenic acid) |
| | water for injection |
| 0.9 wt. % NaCl in H₂O | |
| MMA BonOs ® (AAP | see Example 1 |
| Implantate AG) | |
| PMMA BonOs ® (AAP | see Example 1 |
| Implantate AG) | |

The preparation of cement mixture and samples was carried out as in Example 1, with the exception that 0.5 molar gadobenic acid dimeglumine salt (Multihance®, Bracco Altana Pharma GmbH) was used as contrast agent. The components of the mixture are summarized in Table 2. The final samples contained between 0 wt. % or 0 μmol/g (sample A) and 0.12·10⁻³ wt. % or 7.4 μmol/g (sample N) gadolinium (Gd), and about 22.7 wt. % H₂O each time.

TABLE 2

| | Mixture I | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Multihance ® | 0 | 5 | 10 | 30 | 50 | 100 | 150 | 200 | 250 | 300 | 400 | 500 | 600 | 700 |
| [μl] | | | | | | | | | | | | | | |
| NaCl 0.9% | 10 | 10 | 10 | 10 | 10 | 10 | 10 | 10 | 10 | 10 | 10 | 10 | 10 | 10 |
| [ml] | | | | | | | | | | | | | | |
| | Mixture II | | | | | | | | | | | | | |
| | 1a | 2b | 3c | 4d | 5e | 6f | 7g | 8h | 9i | 10j | 11k | 12l | 13m | 14n |
| Mixture I | 1 | 2 | 3 | 4 | 5 | 6 | 7 | 8 | 9 | 10 | 11 | 12 | 13 | 14 |
| 5 ml | | | | | | | | | | | | | | |
| BonOs ® | 5 | 5 | 5 | 5 | 5 | 5 | 5 | 5 | 5 | 5 | 5 | 5 | 5 | 5 |
| MMA [ml] | | | | | | | | | | | | | | |
| | Sample | | | | | | | | | | | | | |
| | A | B | C | D | E | F | G | H | I | J | K | L | M | N |
| Mixture II | 1a | 2b | 3c | 4d | 5e | 6f | 7g | 8h | 9i | 10j | 11k | 12l | 13m | 14n |
| 10 ml | | | | | | | | | | | | | | |
| BonOs ® | 12 | 12 | 12 | 12 | 12 | 12 | 12 | 12 | 12 | 12 | 12 | 12 | 12 | 12 |
| PMMA [g] | | | | | | | | | | | | | | |
| w(Gd) | 0.00 | 0.89 | 1.79 | 5.34 | 8.89 | 17.7 | 26.4 | 35.0 | 43.6 | 52.1 | 68.7 | 85.1 | 101 | 117 |
| [10⁻³%] | | | | | | | | | | | | | | |
| c(Gd) | 0.00 | 0.06 | 0.11 | 0.34 | 0.57 | 1.13 | 1.68 | 2.23 | 2.77 | 3.31 | 4.37 | 5.41 | 6.43 | 7.43 |
| [μmol/g] | | | | | | | | | | | | | | |

The results are shown in FIG. 2. In T1 FSPGR and T1 SE the samples D through H (0.34 to 2.23 μmol/g Gd) gave the strongest signals. In bFFE the strongest signals were determined in the samples E through K (0.57 to 4.37 μmol/g Gd). In T2 W the samples B and C (0.06 to 0.11 μmol/g Gd) gave the strongest signals.

### EXAMPLE 3

### PMMA Cements Including Varying Concentrations of Mn

### Mangafodipir Trisodium

Chemicals:

| | |
|---|---|
| Teslascan ® | 1 ml contains: |
| (GE Healthcare AS) | 7.57 mg of mangafodipir |
| | trisodium (0.01M) |
| | ascorbic acid |
| | NaCl/NaOH/HCl |
| | Water for injection |
| 0.9 wt. % NaCl in H₂O | |
| MMA BonOs ® (AAP Implantate AG) | see Example 1 |
| PMMA BonOs ® (AAP Implantate AG) | see Example 1 |

The preparation of cement mixture and samples was carried out as in Example 1, with the exception that a 0.01 molar mangafodipir trisodium solution (Teslascan®, GE Healthcare AS) was used as contrast agent. The components of the mixture are summarized in Table 3. The final samples contained between 0 wt. % or 0 μmol/g (sample A) and 0.82·10⁻³ wt. % or 0.15 μmol/g (sample N) manganese (Mn) and about 22.7 wt. % H₂O each time.

TABLE 3

| | Mixture I | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Teslascan ® | 0 | 5 | 10 | 30 | 50 | 100 | 150 | 200 | 250 | 300 | 400 | 500 | 600 | 700 |
| [μl] | | | | | | | | | | | | | | |
| NaCl 0.9% | 10 | 10 | 10 | 10 | 10 | 10 | 10 | 10 | 10 | 10 | 10 | 10 | 10 | 10 |
| [ml] | | | | | | | | | | | | | | |
| | Mixture II | | | | | | | | | | | | | |
| | 1a | 2b | 3c | 4d | 5e | 6f | 7g | 8h | 9i | 10j | 11k | 12l | 13m | 14n |
| Mixture I | 1 | 2 | 3 | 4 | 5 | 6 | 7 | 8 | 9 | 10 | 11 | 12 | 13 | 14 |
| 5 ml | | | | | | | | | | | | | | |
| BonOs ® | 5 | 5 | 5 | 5 | 5 | 5 | 5 | 5 | 5 | 5 | 5 | 5 | 5 | 5 |
| MMA [ml] | | | | | | | | | | | | | | |
| | Sample | | | | | | | | | | | | | |
| | A | B | C | D | E | F | G | H | I | J | K | L | M | N |
| Mixture II | 1a | 2b | 3c | 4d | 5e | 6f | 7g | 8h | 9i | 10j | 11k | 12l | 13m | 14n |
| 10 ml | | | | | | | | | | | | | | |
| BonOs ® | 12 | 12 | 12 | 12 | 12 | 12 | 12 | 12 | 12 | 12 | 12 | 12 | 12 | 12 |
| PMMA [g] | | | | | | | | | | | | | | |
| w(Mn) | 0.00 | 0.01 | 0.01 | 0.04 | 0.06 | 0.12 | 0.18 | 0.24 | 0.30 | 0.36 | 0.48 | 0.59 | 0.71 | 0.82 |
| [10⁻³%] | | | | | | | | | | | | | | |
| c(Mn) | 0.00 | 0.00 | 0.00 | 0.01 | 0.01 | 0.02 | 0.03 | 0.04 | 0.06 | 0.07 | 0.09 | 0.11 | 0.13 | 0.15 |
| [μmol/g] | | | | | | | | | | | | | | |

The results are shown in FIG. 3. While an increase in signal can be recognized in T1 FSPGR and T1 SE, good signal intensities are only achieved in the samples M and N (0.13 to 0.15 μmol/g Mn). The region of quenching characterized by decreasing signal intensity is not reached at the concentrations tested. In bFFE only weak and inhomogeneous signals were observed in all samples, with only slight differences. In T2 W the signal increases in the region of samples B through F (up to 0.02 μmol/g Mn), followed by signal quenching.

It is clear that manganese in the form of the mangafodipir trisodium complex achieves visualization of the bone cement. The appearance in MRT is inhomogeneous as a result of inclusions of fluid and air in the cement.

### EXAMPLE 4

### PMMA Cements Including Varying Concentrations of Fe

### Iron Oxide Nanoparticles

Chemicals:

| | |
|---|---|
| Endorem ® | 8 ml contain: |
| (Guerbet GmbH) | 126.5 mg of superparamagnetic |
| | iron oxide nanoparticles (89.6 |
| | mg of Fe), dextran, anhydrous |
| | citric acid, D-mannitol |
| | water for injection |
| 5 wt. % glucose in H₂O | |
| MMA BonOs ® (AAP Implantate AG) | see Example 1 |
| PMMA BonOs ® (AAP Implantate | see Example 1 |
| AG) | |

The preparation of cement mixture and samples was carried out as in Example 1, with the exception that a suspension of iron oxide nanoparticles (Endorem®, Guerbet GmbH) containing 0.2 mol/l Fe was used as contrast agent and a glucose solution instead of the NaCl solution. The components of the mixture are summarized in Table 4. The final samples contained between 0 wt. % or 0 mmol/g (sample A) and 4.98·10⁻³ wt. % or 0.89 μmol/g (sample N) iron (Fe) and about 22.7 wt. % H₂O each time.

TABLE 4

| | Mixture I | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Endorem ® | 0.0 | 0.1 | 0.5 | 1.0 | 5.0 | 10.0 | 20.0 | 30.0 | 50.0 | 100.0 | 150.0 | 200.0 |
| [μl] | | | | | | | | | | | | |
| Glucose | 10 | 10 | 10 | 10 | 10 | 10 | 10 | 10 | 10 | 10 | 10 | 10 |
| 5% [ml] | | | | | | | | | | | | |
| | Mixture II | | | | | | | | | | | |
| | 1a | 2b | 3c | 4d | 5e | 6f | 7g | 8h | 9i | 10j | 11k | 12l |
| Mixture I | 1 | 2 | 3 | 4 | 5 | 6 | 7 | 8 | 9 | 10 | 11 | 12 |
| 5 ml | | | | | | | | | | | | |
| BonOs ® | 5 | 5 | 5 | 5 | 5 | 5 | 5 | 5 | 5 | 5 | 5 | 5 |
| MMA [ml] | | | | | | | | | | | | |
| | Sample | | | | | | | | | | | |
| | A | B | C | D | E | F | G | H | I | J | K | L |
| Mixture II | 1° | 2b | 3c | 4d | 5e | 6f | 7g | 8h | 9i | 10j | 11k | 12l |
| 10 ml | | | | | | | | | | | | |
| BonOs ® | 12 | 12 | 12 | 12 | 12 | 12 | 12 | 12 | 12 | 12 | 12 | 12 |
| PMMA [g] | | | | | | | | | | | | |
| w(FeO) | 0.000 | 0.003 | 0.013 | 0.025 | 0.127 | 0.254 | 0.507 | 0.759 | 1.263 | 2.514 | 3.752 | 4.978 |
| [10⁻³%] | | | | | | | | | | | | |
| c(FeO) | 0.000 | 0.000 | 0.002 | 0.005 | 0.023 | 0.045 | 0.091 | 0.136 | 0.226 | 0.450 | 0.672 | 0.891 |
| [μmol/g] | | | | | | | | | | | | |

The results are shown in FIG. 4. Good signals in each of the samples F through I (0.045 to 0.226 μmol/g Fe) are visible in T1 FFE and T1 SE. On the other hand, only those samples containing negligible concentrations of the contrast agent give a good signal in bFFE and T2 weighting, for which reason it can be assumed that the iron oxide gives rise to a signal decrease, while the cement can be visualized through the incorporated aqueous glucose solution.

It is clear that an MRT-visible cement can be prepared using iron oxide nanoparticles. However, the appearance in MRT is inhomogeneous as a result of inclusions of fluid and air in the cement.

### EXAMPLE 5

### PMMA Cements Including Varying Concentrations of Mn

### Manganese(II) Chloride Tetrahydrate (MnCl₂×4H₂O)

Chemicals:

| | |
|---|---|
| Solution of contrast agent: | 1 ml contains |
| | 10 mg of manganese(II) chloride |
| | tetrahydrate (0.05M) in H₂O |
| 0.9 wt. % NaCl in H₂O | |
| MMA BonOs ® (AAP Implantate AG) | see Example 1 |
| PMMA BonOs ® (AAP Implantate | see Example 1 |
| AG) | |

The preparation of cement mixture and samples was carried out as in Example 1, with the exception that a self-prepared aqueous 0.05 molar MnCl₂×4H₂O solution was used as contrast agent. The components of the mixture are summarized in Table 5. The final samples contained between 0.03·10⁻³ wt. % or 0.01 μmol/g (sample A) and 4.1·10⁻³ wt. % or 0.74 μmol/g (sample M) manganese (Mn) and about 22.7 wt. % H₂O each time.

TABLE 5

| | Mixture I | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| MnCl_{2•}4H₂O | 0 | 5 | 10 | 30 | 50 | 100 | 150 | 200 | 250 | 300 | 400 | 500 | 600 | 700 |
| [μl] | | | | | | | | | | | | | | |
| NaCl 0.9% | 10 | 10 | 10 | 10 | 10 | 10 | 10 | 10 | 10 | 10 | 10 | 10 | 10 | 10 |
| [ml] | | | | | | | | | | | | | | |
| | Mixture II | | | | | | | | | | | | | |
| | 1a | 2b | 3c | 4d | 5e | 6f | 7g | 8h | 9i | 10j | 11k | 12l | 13m | 14n |
| Mixture I | 1 | 2 | 3 | 4 | 5 | 6 | 7 | 8 | 9 | 10 | 11 | 12 | 13 | 14 |
| 5 ml | | | | | | | | | | | | | | |
| BonOs ® | 5 | 5 | 5 | 5 | 5 | 5 | 5 | 5 | 5 | 5 | 5 | 5 | 5 | 5 |
| MMA [ml] | | | | | | | | | | | | | | |
| | Sample | | | | | | | | | | | | | |
| | A | B | C | D | E | F | G | H | I | J | K | L | M | N |
| Mixture II | 1a | 2b | 3c | 4d | 5e | 6f | 7g | 8h | 9i | 10j | 11k | 12l | 13m | 14n |
| 10 ml | | | | | | | | | | | | | | |
| BonOs ® | 12 | 12 | 12 | 12 | 12 | 12 | 12 | 12 | 12 | 12 | 12 | 12 | 12 | 12 |
| PMMA [g] | | | | | | | | | | | | | | |
| w(Mn) | 0.00 | 0.03 | 0.06 | 0.19 | 0.31 | 0.62 | 0.92 | 1.22 | 1.52 | 1.82 | 2.40 | 2.97 | 3.53 | 4.08 |
| [10⁻³%] | | | | | | | | | | | | | | |
| c(Mn) | 0.00 | 0.01 | 0.01 | 0.03 | 0.06 | 0.11 | 0.17 | 0.22 | 0.28 | 0.33 | 0.44 | 0.54 | 0.64 | 0.74 |
| [μmol/g] | | | | | | | | | | | | | | |

The results are shown in FIG. 5. The samples D through H (0.03 to 0.22 μmol/g Mn) showed good signal intensity in T1 FFE, T1 W SE pre and T1 W TSE, whereas the samples B and C (around 0.01 μmol/g Mn) showed good signal intensity in T2 FSE, which however drops at higher concentrations.

It is clear that an MRT-visible bone cement can be prepared using manganese in the form of the chloride salt. Moreover, the cement samples allow good mixing and are relatively homogeneous even in the cured state.

### EXAMPLE 6

### Comparison of PMMA Cements Including Different MRT Contrast Agents, with and without Addition of Water Each Time

To investigate the influence of water on the visibility of bone cements in MRT, PMMA cements with sole addition of MRT-signaling component and PMMA cements according to the present invention added with water in addition to the MRT-signaling component were prepared. In particular, inventive bone cements according to the compositions of Example 1, sample F (PMMA cement including about 1 μmol/g Gd (Dotarem®) and 23 wt. % H₂O), Example 2, sample F (PMMA cement including about 1 μmol/g Gd (Multihance®) and 23 wt. % H₂O), and Example 5, sample E (PMMA cement including about 1 μmol/g Mn (MnCl₂×4H₂O) and 23 wt. % H₂O) were prepared. Comparative samples (Comparative Examples 1F, 2F, 5E) of identical composition but without water were prepared for each of these samples. Consequently, the comparative samples merely contained the water introduced by the aqueous solution of contrast agent, which showed a negligible effect. As in the examples above, cylindrical test specimens of the cement samples were prepared by filling into disposable syringes. The test specimens of the samples (Ex. 1F, 2F, 5E), together with the comparative samples (Comp. Ex. 1F, 2F, 5E) and a reference sample free of contrast agent and water (sample Z), which was prepared according to the manufacturer's instructions and carried along, were arranged on a screen which is shown in the lower part of FIG. 6. The samples thus arranged were placed in a water bath and measured in open MRT (Philips Panorama 1.0 Tesla) using the sequence T1 W SE. The results are shown in the upper part of FIG. 6. As can be seen, each of the samples according to the invention shows a distinct signal (middle row), whereas the comparative samples free of water (bottom row) as well as the reference sample Z free of contrast agent and water (top) give no signal, but can only be identified through the absence of signals. This result shows that the presence of water, in addition to the MRT-signaling component, is an indispensable precondition for the production of an MRT-signaling cement.

### EXAMPLE OF USE

### Vertebroplasty Using Gd-Containing PMMA Cement Including Varying Concentrations of Gd

### Gadobenic Acid Meglumine

A cement was produced in accordance with Example 1, i.e. using gadobenic acid meglumine (Dotarem® Guerbet GmbH) as MRT contrast agent. Human cadaver spines were drilled and the boreholes filled with a conventional cement prepared according to the manufacturer's instructions or with the Gd-containing cement according to the invention. Measurements were carried out in a 1.5 Tesla Gyro Scan MRI (Philips Medical Systems) using different sequences.

FIG. 7 shows a cross-sectional representation a vertebral body with a filling of conventional PMMA bone cement (left) and PMMA bone cement according to the invention (right). While the conventional cement gives no signal (arrow a), the cement according to the invention can be recognized through a positive signal (arrow b) in the T1 sequence as shown. The same result can be seen in the sagittal representation in the T2 sequence in FIG. 8 on the left. The positive effect is even more clearly visible in the T1 sequence (FIG. 7, right) wherein the cement according to the invention gives a bright signal contrasting with the otherwise dark background, while the conventional cement at best can be identified through the absence of a signal.

1. A bone cement mixture for producing an MRT-signaling bone cement, said mixture containing
(a) at least one type of polymerizable organic monomers and optionally at least one already polymerized polymer component;
(b) at least one MRT-signaling component with a magnetic susceptibility and a concentration in the cement mixture sufficient to generate a visible signal in at least one selected MRT sequence; and
(c) at least 1 wt. % water.

2. The MRT cement mixture according to claim 1,
characterized in that
the water is included at a mass fraction of from 10 to 60 wt. %, in particular from 5 to 45 wt. %, preferably from 14 to 23 wt. %, based on the total mass of the mixture.

3. The MRT cement mixture according to claim 1,
characterized in that
the concentration of said at least one MRT-signaling component is dimensioned such that the cement is visible at least in a T1 sequence or a T1-based sequence.

4. The MRT cement mixture according to claim 1,
characterized in that
the at least one MRT-signaling component comprises a paramagnetic or ferromagnetic metal which is in metallic form, in the form of a compound, salt and/or complex.

5. The MRT cement mixture according to claim 1,
characterized in that
the at least one MRT-signaling component is selected in particular from transition metals, lanthanides and alkaline earth metals as well as compounds, salts and complexes thereof.

6. The MRT cement mixture according to claim 5,
characterized in that
the at least one MRT-signaling component is selected from the group comprising manganese, iron, cobalt, nickel, copper, chromium, titanium, vanadium, zinc, scandium, gadolinium, dysprosium, magnesium and calcium, in the form of metals or alloys, compounds, complexes or salts.

7. The MRT cement mixture according to claim 5,
characterized in that
the metal of the at least one MRT-signaling component has a concentration of from 0.05 to 5 μmol/g, particularly from 0.1 to 3 μmol/g, based on the total mass of the cement mixture.

8. The MRT cement mixture according to claim 1,
characterized in that
the at least one MRT-signaling component is present in the cement mixture in the form of particles or as a dispersion or solution.

9. The MRT cement mixture according to claim 1,
characterized in that
the at least one type of polymerizable organic monomers is selected from the group of acrylates, in particular comprising methacrylate, methyl methacrylate, ethyl acrylate, ethyl methacrylate, propyl acrylate, propyl methacrylate, butyl acrylate and butyl methacrylate.

10. The MRT cement mixture according to claim 1,
characterized in that
the at least one already polymerized polymer component is an acrylate-based polymer or copolymer, in particular based on acrylates comprising methacrylate, methyl methacrylate, ethyl acrylate, ethyl methacrylate, propyl acrylate, propyl methacrylate, butyl acrylate and butyl methacrylate.

11. The MRT cement mixture according to claim 1,
characterized in that
the cement mixture includes at least one initiator and/or activator to induce polymerization of the monomers.

12. The MRT cement mixture according to claim 1,
characterized in that
the cement mixture is in the form of a kit in separate components, in particular in at least one liquid phase containing the monomers and a solid phase containing the polymer component.

13. The MRT cement mixture according to claim 12,
characterized in that
the kit comprises two liquid phases, with a first liquid phase containing the monomers and a second liquid phase containing said at least one MRT-signaling component and the water.

14. An MRT-signaling bone cement produced from a bone cement mixture according to claim 1.
